(19) United States
(12) Patent Application Publication
(10) Pub. No.: US 2018/0036504 A1
(43) Pub. Date: Feb. 8, 2018

(54) OXYGEN DELIVERY SYSTEM

(71) Applicant: Willis Tackett, Virgie, KY (US)

(72) Inventor: Willis Tackett, Virgie, KY (US)

(21) Appl. No.: 15/651,338

(22) Filed: Jul. 17, 2017

Related U.S. Application Data
(60) Provisional application No. 62/370,090, filed on Aug. 2, 2016.

(51) Int. Cl. A61M 16/06; A62B 7/02; A45F 5/02; A61M 16/10

(52) U.S. Cl. CPC A61M 16/0672; A61M 16/1005; A62B 7/02; A45F 5/021; A45F 5/02; A61M 2202/0208; A61M 2209/088

(57) ABSTRACT

An oxygen deliver system for supplying oxygen from a supply tank under pressure to a patient wearing a nasal cannula is provided. The system includes a first tube connected at a first end to the oxygen supply tank for receiving oxygen therefrom, a swivel connected at a first end to a second end of the first tube, a second tube connected at a first end to a second end of the swivel and connected at a second end to the nasal cannula for delivering the oxygen to the patient, and a receiver for limiting movement of the swivel such that the first and second tubes are retained in position and allowed to revolve around the swivel.

### TECHNICAL FIELD

This document relates generally to patient oxygen supply systems, and more specifically to a system for delivering a patient's oxygen supply.

### BACKGROUND

Nasal cannulas include flexible tubing used to supply oxygen to the nasal passages of a patient in both home and hospital settings. Typically an oxygen supply tank is connected to an extended patient oxygen supply tube which terminates in the nasal cannulas for supplying pressurized oxygen to the patient. It is important in most instances that the supply of oxygen be continuous and not hindered by occlusion or pinching of the oxygen supply tube. Such pinching and occlusion can often result from movement of the patient given the fact that the nasal cannulas are fixed to the patient's head.

The present invention provides for an oxygen supply system utilizing a rotatable tube coupling device that joins a supply tube from the oxygen supply tank to a second tube connected to the nasal cannulas. This allows the oxygen supply tubes to rotate freely relative one another to allow for patient movement without inadvertent removal of the nasal cannula or occlusion of the oxygen supply tubes.

### SUMMARY OF THE INVENTION

In accordance with the purposes and benefits described herein, an oxygen delivery system for supplying oxygen from a supply tank under pressure to a patient wearing a nasal cannula may be broadly described as comprising a first tube connected at a first end to the oxygen supply tank for receiving oxygen therefrom, a swivel connected at a first end to a second end of said first tube, a second tube connected at a first end to a second end of said swivel and connected at a second end to the nasal cannula for delivering the oxygen to the patient, and a receiver for limiting movement of the swivel, whereby the first and second tubes are retained in position and allowed to revolve around the swivel.

In another possible embodiment, the receiver attaches to a belt of the patient. In another, the receiver loops around a belt of the patient. In yet another, the receiver attaches to an article of clothing worn by the patient. In still another, the receiver attaches to a piece of furniture supporting the patent.

In one other possible embodiment, the receiver includes a ring at a distal end for receiving said swivel. In another, the receiver attaches to a belt of the patient.

In still another possible embodiment, the receiver includes an elongated piece of material and a fastener for securing a first end of said material to a second end of said material, adjacent said ring, forming a loop.

In yet another possible embodiment, the loop is sized to receive a belt worn by the patient.

In another possible embodiment, the loop receives a clip for securing the receiver to the patient or a piece of furniture supporting the patient.

In still another possible embodiment, the elongated piece of material is leather. In another, the fastener is a snap.

In one additional embodiment, an oxygen deliver system for supplying oxygen from a supply tank under pressure to a nasal cannula includes a first tube coupled at a first end to the oxygen supply tank for receiving oxygen therefrom, a mechanism coupled at a first end to a second end of the first tube a second tube coupled at a first end to a second end of the mechanism and coupled at a second end to the nasal cannula for delivering the oxygen to the patient, and a receiver for limiting movement of the mechanism, whereby the mechanism coupled to the first and second tubes enables one of the first and second tubes to revolve without turning the other.

In the following description, there are shown and described several embodiments of an oxygen delivery system for supplying oxygen from a supply tank under pressure to a patient wearing a nasal cannula. As it should be realized, the systems are capable of other, different embodiments and their several details are capable of modification in various, obvious aspects all without departing from the methods and assemblies as set forth and described in the following claims. Accordingly, the drawings and descriptions should be regarded as illustrative in nature and not as restrictive.

### BRIEF DESCRIPTION OF THE DRAWING FIGURES

The accompanying drawing figures incorporated herein and forming a part of the specification, illustrate several aspects of the oxygen delivery system and together with the description serve to explain certain principles thereof. In the drawing figures:

FIG. 1 is a rear view of a first embodiment of a receiver;

FIG. 2 is a front view of the first embodiment of the receiver;

FIG. 3 is a rear view of a second embodiment of a receiver supported by a belt;

FIG. 4 is a front view of the second embodiment of the receiver;

FIG. 5 is a rear view of a third embodiment of a receiver;

FIG. 6 is a front view of the third embodiment of the receiver;

FIG. 7 illustrates a receiver clipped to a belt loop of a patient; and

FIG. 8 illustrates a receiver coupled to a swivel connecting first and second tubes.

Reference will now be made in detail to the present preferred embodiments of the oxygen delivery system, examples of which are illustrated in the accompanying drawing figures, wherein like numerals are used to represent like elements.

### DETAILED DESCRIPTION

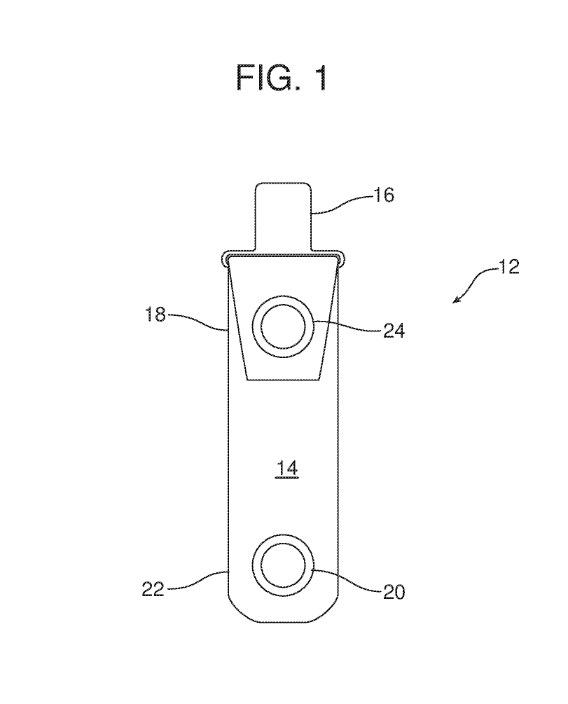

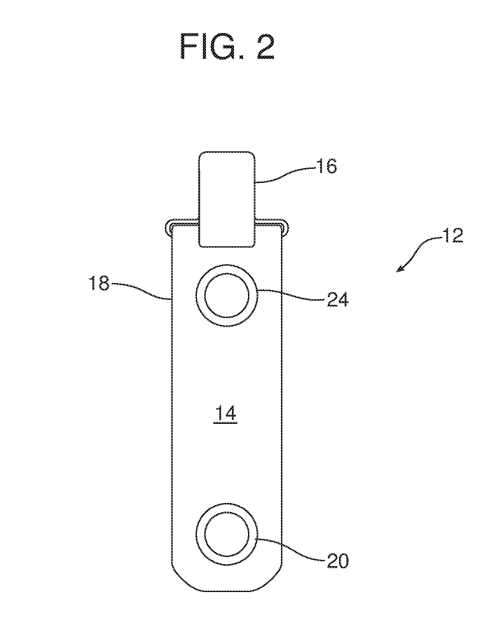

Reference is now made to FIGS. 1 and 2 which show front and rear views of a receiver 12 of an oxygen delivery system 10. The receiver 12 includes an elongated piece of material 14 looped through a clip 16 at a first end 18 and a ring 20 positioned adjacent a second, distal end 22 of the receiver. In the described embodiment, the material is a soft leather material although other materials may be utilized.

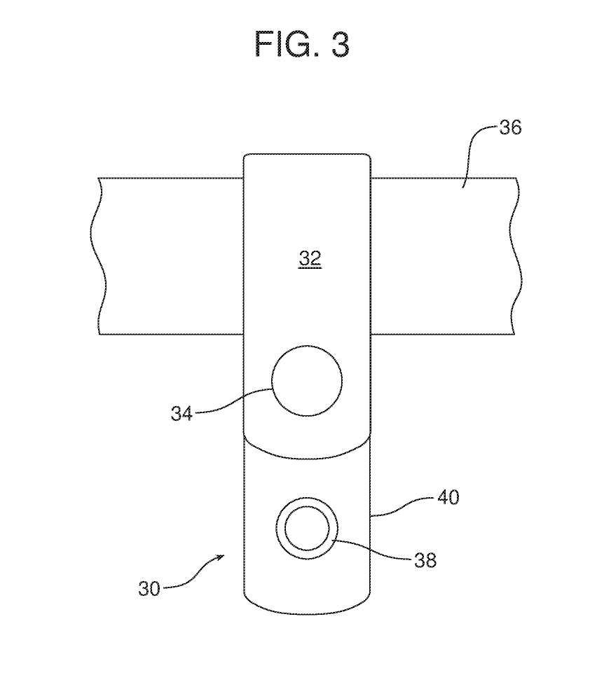

The leather material 14 is looped through and secures the clip 16 adjacent the first end 18 of the receiver 12. The looped material is fastened to itself utilizing a snap 24 in the described embodiment. The clip 16 is an alligator type clip for clasping a belt loop 26 (shown in FIG. 3) of the patient. Alternatively, the clip can be attached to a shirt or other article of clothing, or even a piece of furniture supporting the patient. For example, the clip could be attached to an arm of a chair, a wheel chair, or a bed, or linens or blankets on the bed.

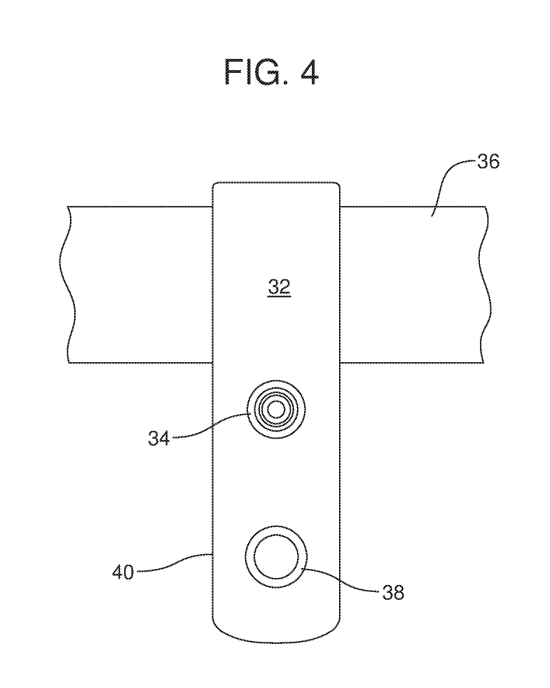

In a second embodiment, shown in FIGS. 3 and 4, a receiver 30 includes an elongated piece of material 32 formed into a loop and fastened to itself utilizing a snap 34. The loop is sufficiently sized to receive a belt 36 worn by the patient for supporting the receiver 30. Again, a ring 38 is positioned adjacent a second, distal end 40 of the receiver 30 and the material is a soft leather material.

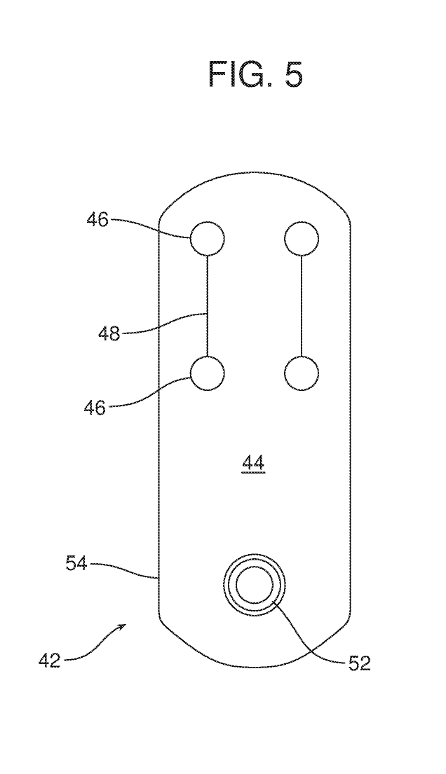

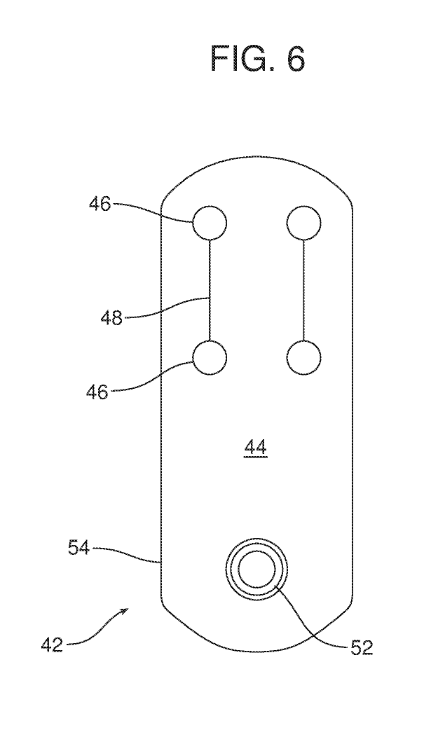

In a third embodiment, shown in FIGS. 5 and 6, a receiver 42 includes an elongated piece of material 44 having apertures 46 forming end points of a pair of slits 48, 50 or slots cut or formed in the material for receiving a belt (not shown) worn by the patient for supporting the receiver 42. In the described third embodiment, the belt is fed into a first slot 49 and exits through a second slot 50 thereby securing the receiver 42 to the patient's belt. The receiver 42 again includes a ring 52 positioned adjacent a second, distal end 54 of the receiver for securing a swivel, or mechanism coupling first and second tubes and enabling one of them to revolve without turning the other, to the receiver.

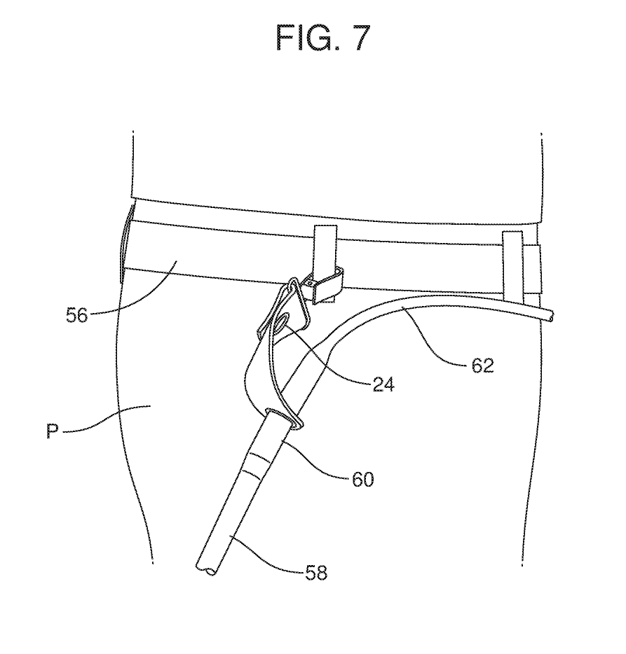

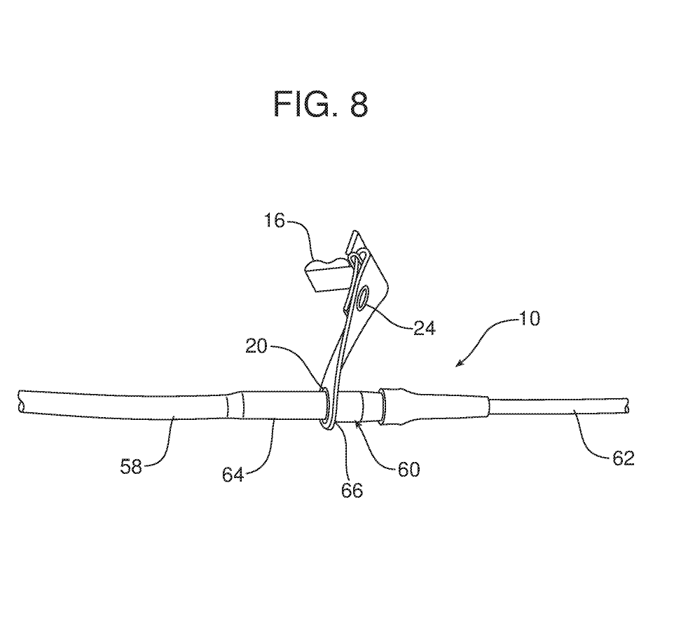

With reference to FIG. 7, the first embodiment of the receiver 12 is shown attached to a belt 56 of a patient P. The receiver 12 is looped around the belt 56 and held in position using the snap 24 in the described embodiment. As described above, the receiver can be fastened to itself utilizing any suitable fastener. As shown, a first tube 58 extends rearward for attachment to an oxygen supply tank (not shown). Oxygen from the tank is fed through the first tube 58, under pressure, to a swivel 60 where the first tube is coupled, or attached, to a first end of the swivel. The swivel 60, or other mechanism, couples the first tube 58 and a second tube 62 enabling one of them to revolve without turning the other. As best shown in FIG. 8, the first tube 58 may include a threaded female fitting 64 for receiving a mating threaded male fitting 66 of the swivel 60. Alternatively, the first tube may be press fitted into the swivel or other means known in the art may be utilized to secure the first tube to the swivel.

As further shown in FIGS. 7 and 8, the swivel 60 extends through the ring 20 of the receiver 12 which holds the swivel and attached tubing 58, 62 in position. As shown in FIG. 7, the receiver 12 is maintained in position adjacent the patient's hip in the described embodiment. Alternately, the receiver may be attached to clothing of the patient P, a piece for furniture upon which the patient rests, or to blankets or sheets associated with the furniture. For example, the clip 16 may be attached to sheets or a blanket on a bed supporting the patient, or to a material covering an arm of a chair, wheelchair, or couch supporting the patient.

As best shown in FIG. 8, the second tube 62 is coupled, or attached, at a second end of the swivel 60 as described above with regard to the first tube 58. The oxygen from the tank is fed through the swivel 60 and into the coupled second tube 62 where it travels, under pressure, to the patient's nasal cannula (not shown). By securing the first and second tube ends at the swivel 60, the tubes 58, 62 are retained in position yet allowed to revolve or rotate around the swivel for ease of movement of the patient P.

In summary, numerous benefits result from the oxygen deliver system 10 for supplying oxygen from a supply tank under pressure to a patient wearing a nasal cannula as illustrated in this document. The foregoing has been presented for purposes of illustration and description. It is not intended to be exhaustive or to limit the embodiments to the precise form disclosed. Obvious modifications and variations are possible in light of the above teachings. All such modifications and variations are within the scope of the appended claims when interpreted in accordance with the breadth to which they are fairly, legally and equitably entitled.

What is claimed:

1. An oxygen deliver system for supplying oxygen from a supply tank under pressure to a patient wearing a nasal cannula, comprising:
a first tube connected at a first end to the supply tank for receiving oxygen therefrom;
a swivel connected at a first end to a second end of said first tube;
a second tube connected at a first end to a second end of said swivel and connected at a second end to the nasal cannula for delivering the oxygen to the patient; and
a receiver for limiting movement of the swivel,
whereby the first and second tubes are retained in position and allowed to revolve around the swivel.

2. The oxygen deliver system of claim 1, wherein said receiver attaches to a belt of the patient.

3. The oxygen deliver system of claim 1, wherein said receiver loops around a belt of the patient.

4. The oxygen deliver system of claim 1, wherein said receiver attaches to an article of clothing worn by the patient.

5. The oxygen deliver system of claim 1, wherein said receiver attaches to a piece of furniture supporting the patent.

6. The oxygen deliver system of claim 1, wherein said receiver includes a ring at a distal end for receiving said swivel.

7. The oxygen deliver system of claim 6, wherein said receiver attaches to a belt of the patient.

8. The oxygen deliver system of claim 6, wherein said receiver includes an elongated piece of material and a fastener for securing a first end of said material to a second end of said material, adjacent said ring, forming a loop.

9. The oxygen deliver system of claim 8, wherein said loop is sized to receive a belt worn by the patient.

10. The oxygen deliver system of claim 8, wherein said loop receives a clip for securing the receiver to the patient or a piece of furniture supporting the patient.

11. The oxygen deliver system of claim 8, wherein said elongated piece of material is leather.

12. The oxygen deliver system of claim 8, wherein said fastener is a snap.

13. An oxygen deliver system for supplying oxygen from a supply tank under pressure to a nasal cannula, comprising:
a first tube coupled at a first end to the supply tank for receiving oxygentherefrom;
a mechanism coupled at a first end to a second end of said first tube;
a second tube coupled at a first end to a second end of said mechanism and coupled at a second end to the nasal cannula for delivering the oxygen to the patient; and
a receiver for limiting movement of said mechanism,
whereby said mechanism coupled to said first and second tubes enables one of said first and second tubes to revolve without turning the other.

14. The oxygen deliver system of claim 13, wherein said receiver attaches to a belt of the patient.

15. The oxygen deliver system of claim 13, wherein said receiver loops around a belt of the patient.

16. The oxygen deliver system of claim 13, wherein said rece attaches to an article of clothing worn by the patient.

17. The oxygen deliver system of claim 13, wherein said receiver attaches to a piece of furniture supporting the patent.

18. The oxygen deliver system of claim 13, wherein said receiver includes a ring at a distal end for receiving said mechanism.

19. The oxygen deliver system of claim 18, wherein said receiver attaches to a belt of the patient.

20. The oxygen deliver system of claim 18, wherein said receiver includes an elongated piece of material and a fastener for securing a first end of said material to a second end of said material, adjacent said ring, forming a loop.
